(19) United States
(12) Plant Patent Application Publication
(10) Pub. No.: US 2016/0316600 P1
(43) Pub. Date: Oct. 27, 2016

(54) Thuja plant named 'Lesdasma'

(50) ;

(71) Applicant: Jan Lescow, Bokholt-Hanredder (DE)

(72) Inventor: Jan Lescow, Bokholt-Hanredder (DE)

(21) Appl. No.: 14/545,325

(22) Filed: Apr. 22, 2015

(51) Int. Cl. A01H 7/00

(52) U.S. Cl. PLT/213

(57) ABSTRACT

A new Thuja plant particularly distinguished by having dark green fall to winter leaf color, dense branching and a slender conical plant habit, is disclosed.

### CROSS REFERENCE TO RELATED APPLICATION

Plant Breeder's Rights for ‘Lesdasma’ were applied for in the European Community on Nov. 4, 2011, Application No. 2011/2655. ‘Lesdasma’ has not been made publicly available or sold more than one year prior to the priority date of this application.

### GENUS AND SPECIES

Thuja occidentalis

### VARIETY DENOMINATION

‘Lesdasma’

### BACKGROUND OF THE NEW PLANT

The present invention comprises a new and distinct variety of Thuja, botanically known as Thuja occidentalis and hereinafter referred to by the variety name ‘Lesdasma’. ‘Lesdasma’ originated from a whole plant mutation of the commercial Thuja variety ‘Smaragd’ (patent status unknown).

In approximately May of 1998, a whole plant mutation of Thuja variety ‘Smaragd’ was discovered in Bokholt-Hanredder, Germany. The whole plant mutation was selected in approximately September of 2002 in Bokholt-Hanredder, Germany and grown for evaluation. The mutation was subsequently named ‘Lesdasma’. In September, 2004, ‘Lesdasma’ was first vegetatively propagated by cuttings in Bokholt-Hanredder, Germany. ‘Lesdasma’ was found to reproduce true to type in successive generations of asexual propagation via cuttings.

### SUMMARY

The following are the most outstanding and distinguishing characteristics of this new variety when grown under normal horticultural practices in Aarslev, Denmark.
1. Dark green fall to winter leaf color;
2. Dense branching; and
3. Slender conical plant habit.

### DESCRIPTION OF THE PHOTOGRAPHS

This new Thuja plant is illustrated by the accompanying photographs, which shows plant grown in soil and then potted. The photographs are of an eight (8) year-old plant grown outdoors in Bokholt-Hanredder, Germany in October 2011. The colors shown are as true as can be reasonably obtained by conventional photographic procedures.

FIG. 1 shows the cultivar Thuja ‘Lesdasma’ grown in a pot.

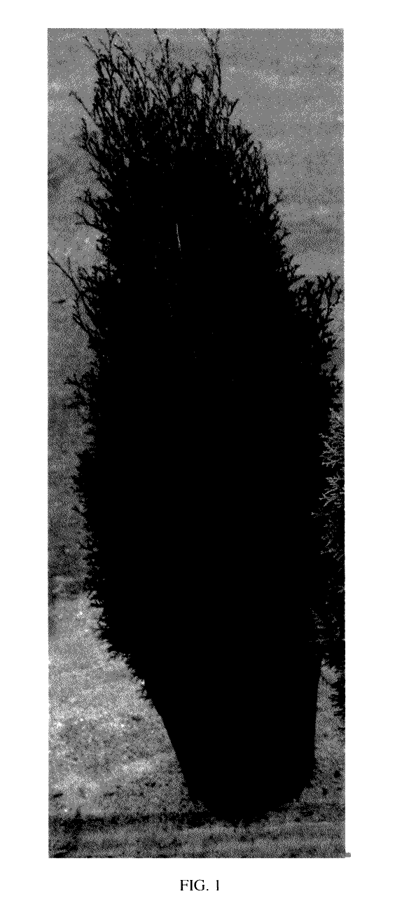

FIG. 2 shows FIG. 1 expanded to show a conventional Thuja ‘Smaragd’ on the right and the cultivar Thuja ‘Lesdasma’ on the left.

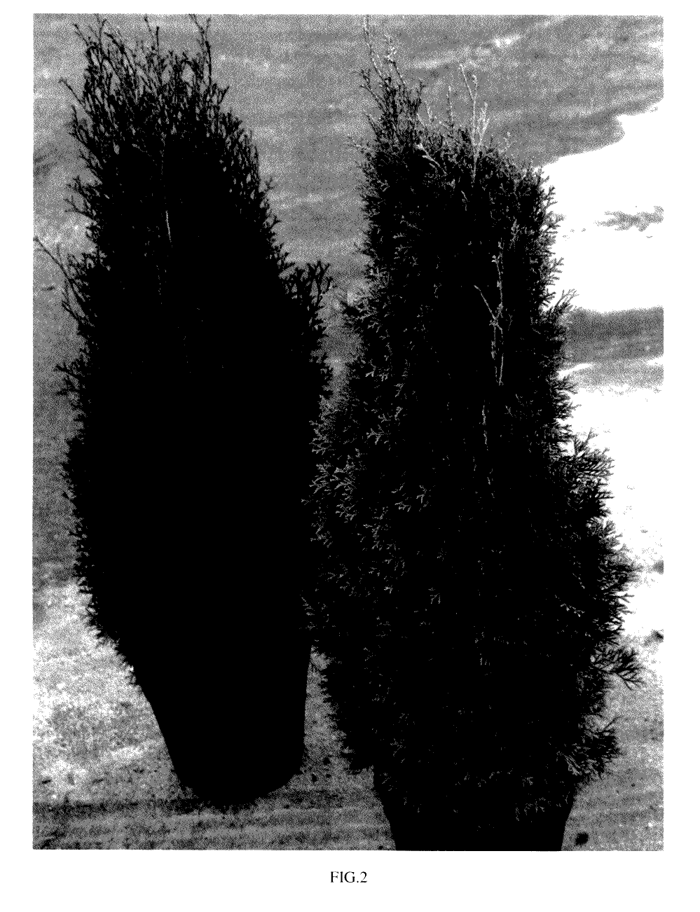

### DESCRIPTION OF THE NEW VARIETY

The following detailed descriptions set forth the distinctive characteristics of ‘Lesdasma’. The data which define these characteristics were collected from asexual reproductions carried out in Aarslev, Denmark. Data was collected on plants 3 to 6 years of age. Color references are to The R.H.S. Colour Chart of The Royal Horticultural Society of London (R.H.S.), 5^{th} edition (2007).
Classification:
   Family.—Cupressaceae.
   Common name.—Thuja.
   Species.—occidentalis.
   Common name.—Arborvitae, American Arborvitae, Northern Whitecedar.
Parentage:
   Mutation parent.—The commercial line Thuja ‘Smaragd’ (patent status unknown).
Plant:
   Type.—Conifer.
   Propagation type.—Cutting.
   Shape.—Slender conic.
   Density.—Loose to medium.
   Growth rate.—Slow to medium.
   Use.—Thuja occidentalis ‘Lesdasma’ is a medium growing coniferous shrub that can be used as a solitary garden plant or grouped as a hedge planting.
   Height (8 year-old plant).—85.0 cm.
   Width (spread) (8 year-old plant).—38.0 cm.
   Vigor.—Medium.
   Average growth rate.—15.0 cm per season.
   Time to initiate roots during the summer.—Approximately 60 days.
   Number of days to produce a rooted young plant during the summer.—270 days.
   Time to a finished plant.—24 months.
Roots:
   Density.—Densely growing, fine roots.
   Branching.—Dense.
   Thickness.—Fine.
   Texture.—Similar to Thuja occidentalis.
Stem (trunk):
   Diameter.—1.5 cm to 2.0 cm (measured at 5.0 cm from soil).
   Texture.—Smooth, slightly rough.
Lateral branches:
   Branching habit.—Loose to medium in density.
   Type.—Non-monstrous.
   Attitude.—Erect.
   Number of lateral branches per plant (average).—Medium to many.
   Diameter (average).—0.4 cm.
   Length of internode (average).—4.0 cm.
   Texture.—Smooth.
   Strength.—Flexible.
   Pubescence.—Absent.
Leaves:
   Arrangement.—Opposite and simple.
   Type.—Non-linear.
   Fragrance.—Absent.
   Shape.—Rounded.
   Length.—Short to medium.
   Width.—Narrow to medium.
   Thickness.—Thin.
   Axis.—Straight.
   Apex.—Acute.
   Attitude of spray.—Oblique.
   Color.—In spring: Upper side: Green Lower side: Green In early summer: Upper side: Green Lower side: Green In mid-summer to winter: Upper side: Dark Green Lower side: Dark Green.
   Variegation.—Absent.
   Durability to stresses.—High, tolerates urban climates.
   Venation.—None.
   Glands.—Absent or few.
   Glossiness.—Weak.
Cone and seed production: Not observed
Disease and insect resistance: More resistant to fungal diseases of the leaves.

### COMPARISON WITH PARENTAL LINE AND KNOWN VARIETY

‘Lesdasma’ is distinguished from its parent as described in Table 1:

TABLE 1
Comparison with Parental Line and Known Variety

| Trait | ‘Lesdasma’ | ‘Smaragd’ |
|---|---|---|
| Leaf color (mid- | Dark green | Light green |
| summer/fall/winter) | | |
| Growth (branches) | Dense | Very dense |
| Habit | Slender conic | Conic |

I claim:

1. A new and distinct variety of Thuja plant named ‘Lesdasma’ as illustrated and described herein.
